(19) United States
(12) Patent Application Publication
(10) Pub. No.: US 2011/0318796 A1
(43) Pub. Date: Dec. 29, 2011

(54) METHOD FOR EXTRACTING SOLUBLE SUGAR MOLECULES FROM BIOMASS MATERIAL

(75) Inventor: David C. Walther, Oakland, CA (US)

(73) Assignee: Cobalt Technologies, Inc., a Delaware Corporation, Mountain View, CA (US)

(21) Appl. No.: 13/163,446

(22) Filed: Jun. 17, 2011

Related U.S. Application Data
(60) Provisional application No. 61/358,221, filed on Jun. 24, 2010.

(51) Int. Cl. C12P 7/10; C12P 7/36; C12P 7/16; C13K 1/02; C13K 13/00

(52) U.S. Cl. 435/151; 127/37; 127/29; 435/160; 435/165

(57) ABSTRACT

Methods are provided for preparing a hydrolysate containing soluble sugar molecules from biomass that contains cellulose and hemicellulose. Hemicellulose sugars are extracted in the process, and the resulting hydrolysate may be used to support microbial fermentation to produce products of interest.

### CROSS-REFERENCE TO RELATED APPLICATIONS

This application claims the benefit of U.S. Provisional Application No. 61/358,221, filed on Jun. 24, 2010, which is incorporated herein by reference in its entirety.

### FIELD OF THE INVENTION

The invention relates to a method for extracting soluble sugar molecules from biomass material, optionally with deconstruction of residual cellulose, and compositions prepared by such methods.

### BACKGROUND OF THE INVENTION

Many useful products may be produced by microorganisms grown in culture. A carbon source for such cultures is often provided by hydrolysis of cellulosic biomass materials. Soluble sugar molecules released by hydrolysis may be used to support microbial growth. Hydrolysis of biomass material is often hindered due to the structural nature of the material, which limits access of a catalyst, such as acid, to the polymeric carbohydrate substrate molecules. New methods are needed that enhance the extractability of soluble sugar molecules from biomass material.

### BRIEF SUMMARY OF THE INVENTION

Methods for deconstructing biomass and extracting soluble sugar molecules are provided. Methods for use of the resulting hydrolysate in downstream processes for production of bioproducts are also provided.

In one aspect, a method is provided for deconstructing biomass that contains cellulose and hemicellulose for the extraction of sugar molecules from the biomass. In one embodiment, a method is provided for deconstructing and extracting sugar molecules from biomass, including: (a) mechanically disintegrating the biomass in the presence of water and under a first pressure, thereby producing liquid and/or vapor and solid disintegrated biomass; (b) separating the liquid and/or vapor from the solid disintegrated biomass, wherein step (b) may be performed after or in conjunction with step (a); (c) contacting the disintegrated biomass with acid in an amount sufficient to depolymerize a polymeric carbohydrate component of the biomass, thereby producing acid impregnated disintegrated biomass; (d) feeding the acid impregnated disintegrated biomass into a digestor through a pressure changing device, wherein the acid impregnated disintegrated biomass is heated under a second pressure in the digestor at a temperature and for an amount of time sufficient to permit the depolymerization reaction to occur, thereby producing a composition that contains a liquid hydrolysate and residual solids; and (e) separating solids from liquids to produce a liquid hydrolysate and residual solids, wherein the hydrolysate contains soluble hemicellulose sugar molecules and the residual solids contain cellulosic fiber.

In one embodiment, the method is performed with an acid concentration in step (c), a residence time in step (d), and a temperature in step (d) sufficient to produce a liquid hydrolysate that contains hemicellulose sugar molecules and residual solids that contain cellulosic fiber that is at least about 0.35 or 0.37 mm in length. In some embodiments, the acid is nitric acid at a concentration of about 0.1% (w/w) to about 0.5% (w/w). In some embodiments, the digestor is operated at a second pressure of about 90 to about 110 psig, a temperature of about 167° C. to about 176° C. and a residence time of about 3 to about 20, about 8 to about 20, or about 5 to about 10 minutes.

In another embodiment, the method described above is performed with an acid concentration is performed with an acid concentration in step (c), a residence time in step (d), and a temperature in step (d) sufficient to produce a hydrolysate that contains hemicellulose sugars and residual solids that contain cellulosic fiber that is less than about 0.35, 0.30, or 0.28 mm in length. In some embodiments, the cellulosic fiber is less than about 0.35, 0.30, or 0.28 mm in length but is insoluble. In one embodiment, the residual solids do not contain visible cellulosic fiber. In some embodiments, the acid concentration in step (c) is about 1% (w/w) to about 1.5% (w/w), the residence time in step (d) is about 5 minutes to about 10 minutes, and the temperature in step (d) is about 160° C. to about 180° C., or about 120° C. to about 180° C. In some embodiments, the acid is nitric acid at a concentration of about 0.05% (w/w) to about 4% (w/w). In some embodiments, the digestor is operated at a pressure of about 90 to about 110 psig, a temperature of about 160° C. to about 180° C. or about 120° C. to about 180° C., and a residence time of about 4 to about 15 min.

In some embodiments of any of the above methods, the liquid and/or vapor that is separated from solid disintegrated biomass in step (b) comprises extractives.

In some embodiments of any of the above methods, mechanical disintegrating is performed in a thermo-mechanical disintegrator. The thermo-mechanical disintegrator may be selected from, for example, a modular screw device, an oil press, and a screw press. Mechanical disintegration may be performed at a pressure and residence time sufficient to shear apart the biomass to make it accessible for acid-catalyzed depolymerization of carbohydrate polymers. In some embodiments, the first pressure is about 5 to about 50 psig and the residence time is about 5 to about 60 seconds.

In some embodiments, the digestor is operated under a second pressure of about 50 to about 150 psig. In some embodiments, the second pressure is higher than the first pressure.

In some embodiments of any of the above methods, the biomass is contacted with steam prior to mechanical disintegration, which may increase the amount of extractives removed and the degree of disintegration.

In another aspect, a method is provided for deconstructing biomass that contains cellulose and hemicellulose for the extraction of sugar molecules from the biomass, including: (a) contacting the biomass with acid in an amount sufficient to depolymerize a polymeric carbohydrate component of the biomass, thereby producing acid impregnated disintegrated biomass; (b) feeding the acid impregnated disintegrated biomass into a digestor through a pressure changing device, wherein the acid impregnated disintegrated biomass is heated under pressure in said digestor at a temperature and for an amount of time sufficient to permit the depolymerization reaction to occur; and (c) separating solids from liquids to produce a liquid hydrolysate and residual solids, wherein the hydrolysate comprises hemicellulose sugar molecules and the residual solids contain fiber that is less than about 0.35, 0.30, or 0.28 mm in length. In one embodiment, the residual solids do not contain visible cellulosic fiber. In some embodiments, the acid concentration in step (a) is about 0.1% (w/w) to about 5% (w/w), or about 1% (w/w) to about 3% (w/w), the residence time in step (b) is about 8 to about 20 minutes, and the temperature in step (b) is about 160° C. to about 180° C. In some embodiments, the biomass is contacted with steam prior to acid impregnation, which may aid with disintegration of the biomass and extractives removal.

In some embodiments of any of the above methods, the acid is nitric acid.

In some embodiments of any of the above methods, the pressure changing device in the digestor is selected from a plug screw feeder, a rotary valve, or a lockhopper arrangement. In some embodiments, heating of the acid impregnated disintegrated biomass in the digestor is with direct or indirect steam. In some embodiments of any of the above methods, the digestor is operated under pressure. In one embodiment, the digestor is a continuous feed, pressure rated, screw conveyor vessel. In some embodiments, the material that is fed to the digestor comprises a liquid to solid ratio of about 1:1 to about 20:1.

In some embodiments of any of the above methods, solids are separated from liquids to produce a hydrolysate and residual solids in a screw press, belt filter press, centrifuge, settling tank, vacuum filter, sieve screen, or rotary drum dryer.

In some embodiments of any of the above methods, the liquid hydrolysate contains about 10 to about 150 g/l soluble sugar molecules. In some embodiments, the soluble sugar molecules include mannose, xylose, glucose, arabinose, and galactose.

In some embodiments of any of the above methods, the biomass is lignocellulosic material selected from softwood and hardwood, or a combination thereof. In one embodiment, the lignocellulosic material is Lodgepole pine. In one embodiment, the lignocellulosic material is in the form of wood chips.

In another aspect, a liquid hydrolysate containing soluble sugars is provided, prepared according to any of the methods above. In some embodiments, the hydrolysate is conditioned to remove one or more inhibitor(s) of microbial growth and/or production of a bioproduct of interest, thereby producing a conditioned hydrolysate. In some embodiments, conditioning is performed by a process selected from overliming, adsorption, precipitation, ion exchange, steam stripping, enzymatic treatment, evaporation, and filtration, or a combination thereof. A conditioned hydrolysate, produced as described herein, is also provided.

In another aspect, biomass material, e.g., deconstructed biomass material, is provided from which hemicellulose sugars have been extracted according to any of the methods described herein. In some embodiments, the material retains cellulosic fiber that is at least about 0.35 or 0.37 mm in length. In other embodiments, the material contains cellulosic fiber that is less than about 0.35, 0.30, or 0.28 mm in length. In some embodiments, the cellulosic fiber is less than about 0.35, 0.30, or 0.28 mm in length but is insoluble. In one embodiment, the residual solids do not contain visible cellulosic fiber. In one embodiment, the material does not contain or contains very little visible cellulosic fiber.

In another aspect, a method is provided for producing a bioproduct. The method includes culturing a microorganism that produces the bioproduct in a medium that contains a hydrolysate or conditioned hydrolysate, prepared as described herein, under conditions suitable for production of the bioproduct. In one embodiment, the bioproduct is a solvent. In some embodiments, the bioproduct is a biofuel, for example, butanol, ethanol, or acetone, or a combination thereof. In some embodiments, the bioproduct is a biochemical or biochemical intermediate, for example, formate, acetate, butyrate, propionate, succinate, methanol, propanol, and hexanol.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the residual material remaining after performing the hemicellulose extraction procedure described in Example 1 with acid (right hand panel) or water (left hand panel).

### DETAILED DESCRIPTION OF THE INVENTION

The invention provides methods for disintegration and extraction of soluble sugars from biomass material, such as lignocellulosic biomass. The resulting hydrolysates may be used in downstream microbial fermentation processes for production of bioproducts of interest. The methods described herein include acid impregnation of biomass that contains cellulose and hemicellulose, digestion of the acid-impregnated biomass material, and separation of a liquid hydrolysate that contains hemicellulose sugar molecules from residual solid material. Depending on the conditions used in the digestor, the solid material may or may not retain visible cellulosic fiber. For example, the solid material may contain cellulosic fiber that is at least about 0.35 or 0.37 mm in length, or may contain cellulosic fiber that is less than about 0.35, 0.30, or 0.28 mm in length. More harsh digestion conditions result in less cellulose fiber and/or shorter fibers in the solid product. The two types of products may be used for different purposes in downstream processes as described herein.

Advantageously, the methods described herein may reduce the impact of wood extractives on bioproduct, e.g., biofuel, production and process production (i.e., prevents pitch deposits) by reducing or excluding extractives from the hydrolysate produced from a biomass feedstock. The methods described herein may also advantageously permit hydrolysate to be prepared under less harsh process conditions than those described in the art (e.g., reduced acid concentration, reduced temperature). The lower acid concentration and reduced temperature (lower steam pressure) may provide a cost benefit and improve yield. The methods described herein may also advantageously provide a higher productivity (higher throughput, with associated cost benefit) than other known approaches.

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Numeric ranges provided herein are inclusive of the numbers defining the range.

### DEFINITIONS

“A,” “an” and “the” include plural references unless the context clearly dictates otherwise.

“Bioproduct” refers to any substance of interest produced biologically, i.e., via a metabolic pathway, by a microorganism, e.g., in a microbial fermentation process. Bioproducts include, but are not limited to biofuels (e.g., butanol, acetone, ethanol), solvents, biomolecules (e.g., proteins (e.g., enzymes), polysaccharides), organic acids (e.g., formate, acetate, butyrate, propionate, succinate), alcohols (e.g., methanol, propanol, isopropanol, hexanol, 2-butanol, isobutanol), fatty acids, aldehydes, lipids, long chain organic molecules (for example, for use in surfactant production), vitamins, and sugar alcohols (e.g., xylitol).

“Biofuel” refers to fuel molecules (e.g., butanol, acetone, and/or ethanol) produced biologically by a microorganism, e.g., in a microbial fermentation process.

“Biobutanol” refers to butanol (i.e., n-butanol) produced biologically by a microorganism, e.g., in a microbial fermentation process.

“Byproduct” refers to a substance that is produced and/or purified and/or isolated during any of the processes described herein, which may have economic or environmental value, but that is not the primary process objective. Nonlimiting examples of byproducts of the processes described herein include lignin compounds and derivatives, carbohydrates and carbohydrate degradation products (e.g., furfural, hydroxymethyl furfural, formic acid), and extractives (described infra).

“Feedstock” refers to a substance that can serve as a source of sugar molecules to support microbial growth in a fermentation process.

“Deconstruction” refers to mechanical, chemical, and/or biological degradation of biomass to render individual components (e.g., cellulose, hemicellulose) more accessible to further pretreatment processes, for example, a process to release monomeric and oligomeric sugar molecules, such as acid hydrolysis.

“Conditioning” refers to removal of inhibitors of microbial growth and/or bioproduct, e.g., biofuel, production from a hydrolysate produced by hydrolysis of a cellulosic feedstock.

“Titer” refers to amount of a substance produced by a microorganism per unit volume in a microbial fermentation process. For example, biobutanol titer may be expressed as grams of butanol produced per liter of solution.

“Yield” refers to amount of a product produced from a feed material (for example, sugar) relative to the total amount that of the substance that would be produced if all of the feed substance were converted to product. For example, biobutanol yield may be expressed as % of biobutanol produced relative to a theoretical yield if 100% of the feed substance (for example, sugar) were converted to biobutanol.

“Productivity” refers to the amount of a substance produced by a microorganism per unit volume per unit time in a microbial fermentation process. For example, biobutanol productivity may be expressed as grams of butanol produced per liter of solution per hour.

“Wild-type” refers to a microorganism as it occurs in nature.

“Biomass” refers to cellulose- and/or starch-containing raw materials, including but not limited to wood chips, corn stover, rice, grasses, forages, perrie-grass, potatoes, tubers, roots, whole ground corn, grape pomace, cobs, grains, wheat, barley, rye, milo, brans, cereals, sugar-containing raw materials (e.g., molasses, fruit materials, sugar cane, or sugar beets), wood, and plant residues.

“Starch” refers to any starch-containing materials. In particular, the term refers to various plant-based materials, including but not limited to wheat, barley, potato, sweet potato, tapioca, corn, maize, cassava, milo, rye, and brans. In general, the term refers to any material comprised of the complex polysaccharide carbohydrates of plants, comprised of amylose, and amylopectin, with the formula (C₆H₁₀O₅)ₓ, wherein “x” can be any number.

“ABE fermentation” refers to production of acetone, butanol, and/or ethanol by a fermenting microorganism.

“Advanced biofuels” are high-energy liquid transportation fuels derived from low nutrient input/high per acre yield crops, agricultural or forestry waste, or other sustainable biomass feedstocks including algae.

“Lignocellulosic” biomass refers to plant biomass that contains cellulose, hemicellulose, and lignin. The carbohydrate polymers (cellulose and hemicellulose) are tightly bound to lignin.

“Lignins” are macromolecular components of wood that contain phenolic propylbenzene skeletal units linked at various sites.

“Solvent” refers to a liquid or gas produced by a microorganism that is capable of dissolving a solid or another liquid or gas. Nonlimiting examples of solvents produced by microorganisms include n-butanol, acetone, ethanol, acetic acid, isopropanol, n-propanol, methanol, formic acid, 1,4-dioxane, tetrahydrofuran, acetonitrile, dimethylformamide, and dimethyl sulfoxide.

A “protic” solvent contains dissociable H⁺, for example a hydrogen atom bound to an oxygen atom as in a hydroxyl group or a nitrogen atom as in an amino group. A protic solvent is capable of donating a proton (H⁺). Conversely, an “aprotic” solvent cannot donate H⁺.

n-Butanol is also referred to as “butanol” herein.

“Direct steam” refers to steam that is added into a process stream.

“Indirect steam” refers to steam that is not in direct contact with a process fluid, for example, steam that is injected into a jacket or heat exchanger.

“ATCC” refers to the American Type Culture Collection, P.O. Box 1549, Manassas, Va. 20108.

### Feedstock

A feedstock is a substance that provides the base material from which sugar molecules are generated for inclusion in a microbial growth medium, to support the growth of the microorganism. Feedstock used in the methods described herein contains cellulose and hemicellulose. For example, the feedstock may be lignocellulosic biomass or wood pulp.

Cellulose, which is a β-glucan built up of D-glucose units linked by β(1,4)-glycosidic bonds, is the main structural component of plant cell walls and typically constitutes about 35-60% by weight (% w/w) of lignocellulosic materials.

Hemicellulose refers to non-cellulosic polysaccharides associated with cellulose in plant tissues. Hemicellulose frequently constitutes about 20-35% w/w of lignocellulosic materials, and the majority of hemicelluloses consist of polymers based on pentose (five-carbon) sugar units, such as D-xylose and D-arabinose units, hexose (six-carbon) sugar units, such as D-glucose and D-mannose units, and uronic acids such as D-glucuronic acid.

Lignin, which is a complex, cross-linked polymer based on variously substituted p-hydroxyphenylpropane units, typically constitutes about 10-30% w/w of lignocellulosic materials.

Any material containing cellulose and hemicellulose may be used as the feedstock. The material may contain cellulose and hemicellulose with or without lignin.

In some embodiments, the feedstock is woody biomass. In one embodiment, the feedstock is softwood, for example, pine, e.g., Lodgepole or Loblolly pine. In one embodiment, the feedstock contains mountain pine beetle infested pine, for example, dying (“red stage”) or dead (“grey” stage) pine. In another embodiment, the feedstock is hardwood, for example, maple, birch, or ash. In another embodiment, the feedstock is mixed hardwood and softwood. In another embodiment, the feedstock is mixed hardwood. In some embodiments, the woody biomass is in the form of wood chips, sawdust, saw mill residue, wood fines, or a combination thereof.

In some embodiments, the feedstock is obtained as a process stream from a biomass processing facility, for example, a pulp mill. In various embodiments of pulp mill process streams, the process stream may include reject pulp, wood knots or shives, pulp screening room rejects (e.g., essentially cellulose in water), prehydrolysis extraction stream, and/or black liquor.

Lignocellulose contains a mixture of carbohydrate polymers and non-carbohydrate compounds. The carbohydrate polymers contain cellulose and hemicellulose, and the non-carbohydrate portion contains lignin. The non-carbohydrate portion may also contain ash, extractives, and/or other components such as proteins. The specific amounts of cellulose, hemicellulose, and lignin depend on the source of the biomass.

In some embodiments, the feedstock is a lignocellulosic material in the form of wood chips, sawdust, saw mill residue, or a combination thereof. In some embodiments, the lignocellulosic material is from a feedstock source that has been subjected to some form of disease in the growth and/or harvest production period. In one embodiment, the feedstock source is mountain pine beetle infested pine. In another embodiment, the feedstock source is sudden oak death syndrome infested oak, e.g., coastal live oak, tanoak, etc. In another embodiment, the feedstock source is Dutch elm disease infested elm. In other embodiments, the feedstock source is lignocellulosic material that has been damaged by drought or fire.

Lignocellulosic biomass may be derived from a fibrous biological material such as wood or fibrous plants. Examples of suitable types of wood include, but are not limited to, spruce, pine, hemlock, fir, birch, aspen, maple, poplar, alder, salix, cottonwood, rubber tree, marantii, eucalyptus, sugi, and acase. Examples of suitable fibrous plants include, but are not limited to, corn stover and fiber, flax, hemp, cannabis, sisal hemp, bagasse, straw, cereal straws, reed, bamboo, mischantus, kenaf, canary reed, Phalaris arundinacea, and grasses. Other lignocellulosic materials may be used such as herbaceous material, agricultural crop or plant residue, forestry residue, municipal solid waste, pulp or paper mill residue, waste paper, recycling paper, or construction debris. Examples of suitable plant residues include, but are not limited to, stems, leaves, hulls, husks, cobs, branches, bagasse, wood chips, wood pulp, wood pulp, and sawdust.

In some embodiments, a feedstock mix containing about 40% logging residues, about 20% sustainable roundwood, about 20% woody energy crops, and about 20% herbaceous energy crops may be used. This blend can account for regional variation and provide significant flexibility in selecting locations for facilities and in procuring feedstock supply contracts.

In some embodiments, the feedstock contains grass, for example, sugar cane, miscanthus, and/or switchgrass, and/or straw, for example, wheat straw, barley straw, and/or rice straw.

### Methods for Deconstruction of Biomass and Extraction of Hemicellulose Sugar Molecules

Methods are provided herein for deconstruction of feedstock to extract soluble sugar molecules from hemicellulose and optionally cellulose. The methods herein include acid impregnation of biomass that contains cellulose and hemicellulose, digestion of the acid impregnated material, and separation of liquid hydrolysate which contains soluble hemicellulose sugars from residual solid material. Optionally, the method may include mechanical disintegration of the biomass prior to acid impregnation. Optionally, the method may include removal of extractives in conjunction with mechanical disintegration or between mechanical disintegration and acid impregnation of the biomass. In some embodiments, the residual solid material contains cellulosic fiber that is at least about 0.35 or 0.37 mm in length. In other embodiments, the residual solid material contains cellulosic fiber that is less than about 0.35 0.30, or 0.28 mm in length.

In one embodiment, the method includes (a) mechanically disintegrating the biomass in the presence of water and under a first pressure, thereby producing liquid and/or vapor and solid disintegrated biomass; (b) separating liquid and/or vapor from the biomass after or in conjunction with step (a); (c) contacting the disintegrated biomass with acid in an amount sufficient to depolymerize one or more polymeric carbohydrate component(s) (e.g., hemicellulose and optionally some cellulose) of the biomass, thereby producing acid impregnated disintegrated biomass; (d) feeding the acid impregnated disintegrated biomass into a digestor through a pressure changing device, with the impregnated disintegrated biomass heated under a second pressure in the digestor at a temperature and for an amount of time sufficient to permit the depolymerization reaction to occur; and (e) separating solids from liquids to produce a liquid hydrolysate and residual solids.

In one embodiment, the method is performed with an acid concentration is step (c), and at a pressure, temperature, and residence time in step (d) sufficient to produce a hydrolysate that contains hemicellulose sugars and residual solids contains hemicellulose sugar molecules and residual solids contains cellulosic fiber that is at least about 0.35 or 0.37 mm in length.

In another embodiment, the method is performed with an acid concentration in step (c), and at a pressure, temperature, and residence time in step (d) sufficient to produce a hydrolysate that comprises hemicellulose sugars and residual solids that contain cellulosic fiber that is less than about 0.35, 0.30, or 0.28 mm in length.

The acid concentration, residence time in the digestor, temperature in the digestor, and/or pressure in the digestor may be adjusted to produce residual solid material with desired structural properties (e.g., retention or destruction of cellulose fiber). For example, in an embodiment in which the product does not contain or contains very little visible cellulosic fiber, for example, contains cellulosic fiber that is less than about 0.35, 0.30, or 0.28 mm in length, the acid concentration in step (c) may be about 0.05% (w/w) to about 4% (w/w), or about 1% (w/w) to about 1.5% (w/w); the residence time in the digestor may be about 4 to about 15 minutes, or about 5 minutes to about 10 minutes; and the temperature in the digestor may be about 160° C. to about 180° C. or about 60° C. to about 180° C. In one embodiment, the acid concentration in step (c) is about 1% (w/w) to about 1.5% (w/w), the residence time in step (d) is about 5 minutes to about 10 minutes and the temperature in step (d) is about 60° C. to about 180° C. In some embodiments, the acid is nitric acid. In one embodiment, the acid is nitric acid at a concentration of about 0.05% (w/w) to about 4% (w/w). In some embodiments, the second pressure may be about 50 to about 150 psig or about 90 to about 110 psig. In one embodiment, the second pressure is higher than the first pressure. In some embodiments, the second pressure, is about 90 to about 110 psig, the temperature in the digestor is about 120° C. to about 180° C., and the residence time in the digestor is about 4 minutes to about 15 minutes.

In an embodiment in which the product contains cellulosic fiber that is at least about 0.35 or 0.37 mm in length, the acid concentration may be about 0.1% (w/w) to about 0.5% (w/w); the residence time in the digestor may be about 3 to about 20 minutes, about 8 to about 20 minutes, or about 5 to about 10 minutes; and the temperature in the digestor may be about 167° C. to about 176° C. In some embodiments, the acid is nitric acid. In one embodiment, the acid is nitric acid at a concentration of about 0.1% (w/w) to about 0.5% (w/w). In some embodiments, the second pressure may be about 50 to about 150 psig or about 90 to about 110 psig. In one embodiment, the second pressure is higher than the first pressure.

In one embodiment, the method provides production of residual solid material that contains cellulosic fiber that is less than about 0.35, 0.30, or 0.28 mm in length, for example, without or with very little visible cellulosic fiber, without use of mechanical disintegration of the biomass. Such a method may include (a) contacting the biomass with acid in an amount sufficient to depolymerize one or more polymeric carbohydrate component(s) of the biomass (e.g., hemicellulose and optionally some cellulose), thereby producing acid impregnated disintegrated; (b) feeding the acid impregnated disintegrated biomass into a digestor through a pressure changing device, with the acid impregnated biomass heated under pressure in the digestor at a temperature and for an amount of time sufficient to permit the depolymerization reaction to occur, wherein a composition is produced that contains a liquid hydrolysate and residual solids; and (c) separating solids from liquids to produce a liquid hydrolysate and residual solids. The hydrolysate contains hemicellulose sugar molecules and the residual solids contain cellulosic fiber that is less than about 0.35, 0.30, or 0.28 mm in length, for example, without or with very little visible cellulosic fiber. In one embodiment, the acid is nitric acid. In some embodiments, the acid concentration is about 1% (w/w) to about 5% (w/w), or about 1% (w/w) to about 3% (w/w); the residence time in the digestor is about 8 minutes to about 20 minutes; and the temperature in the digestor is about 160° C. to about 180° C. In some embodiments, the digestor is operated under a pressure of about 50 to about 150 psig. In one embodiment, the digestor is operated at a pressure of about 90 to about 110 psig, a temperature of about 160° C. to about 180° C. and a residence time of about 4 to about 15 minutes.

### Deconstruction

In some embodiments, the biomass is deconstructed prior to acid impregnation. Deconstruction may include mechanical disintegration in the presence of water and under pressure, thereby producing liquid and/or vapor and solid disintegrated biomass. In some embodiments, mechanical disintegration may be performed at a pressure and residence time sufficient to shear apart the biomass to render the carbohydrate polymers therein more accessible for acid-catalyzed depolymerization.

In some embodiments, mechanical disintegration is performed in a thermo-mechanical disintegrator. Non-limiting examples of thermo-mechanical disintegrators that may be used in the methods herein include modular screw devices, oil presses, and screw presses.

Mechanical disintegration in the methods herein is typically performed under pressure, for example, a pressure of about 5 to about 50 psig. In some embodiments, the residence time in the disintegrator is about 5 to about 60 seconds.

### Removal of Extractives

In some embodiments, extractives are removed simultaneously with or after mechanical disintegration of the biomass. Extractives are compounds that may be separated in a liquid or vapor phase from the solid disintegrated biomass, and may be inhibitory to a downstream process such as microbial fermentation if present in the biomass hydrolysate.

Non-limiting examples of extractives include terpenes, resin acids, fatty acids, sterols, phenolic compounds, and triglycerides. Extractives may include, but are not limited to, p-coumaric acid, ferulic acid, 4-hydroxybenzoic acid, vanillic acid, syringaldehyde, vanillin, furfural, hydroxymethylfurfural, glucuronic acid, acetic acid, and methanol. Extractives may be removed for other uses, such as production of sterols, or burned to provide energy for a bioproduct production process as described herein.

In some embodiments, mechanical disintegration of the biomass is performed in the presence of water, and extractives are removed when liquid or vapor is separated from the biomass, either during or after the disintegration process. In some embodiments, acetic acid, methanol, and/or terpenes are removed in the vapor phase. In some embodiments, resin acids are removed in the liquid phase.

### Acid Impregnation

The biomass feedstock contains sugar molecules in an oligomeric form, e.g., a polymeric form, which must be hydrolyzed to extract and release soluble monomeric and/or multimeric sugar molecules. The soluble sugar molecules may be converted to bioproduct, in a microbial fermentation as described herein.

In the methods described herein, depolymerization of biomass sugar polymers is catalyzed by acid. The biomass is contacted with acid at a concentration sufficient to depolymerize a polymeric component of the biomass. In some embodiments, the biomass is mechanically disintegrated, as described above, prior to contact with acid.

Acids that may be used for hydrolysis include, but are not limited to, nitric acid, formic acid, acetic acid, phosphoric acid, hydrochloric acid, and sulfuric acid, or a combination thereof. Examples of acid concentrations that may be used in this process are described above, and may be adjusted depending on the desired structure of the product (i.e., length of resulting cellulosic fiber).

In embodiments in which material that retains cellulosic fiber is produced, residual material at the end of the process, may be subjected to acid hydrolysis to release cellulose sugars. In some embodiments, hydrolysis of the residual material is performed at an acid, for example, nitric acid, concentration of about 0.05 to about 0.1%, about 0.1% to about 0.5%, about 0.5% to about 1%, about 1% to about 4%, about 1.3% to about 3.5%, or about 1.3% (w/w) at a temperature of about 190° C. to about 210° C., or about 210° to about 230° C.

### Production of Hydrolysate

The acid-impregnated biomass material is fed into a digestor through a pressure changing device. The material is heated in the digestor at a temperature and for an amount of time sufficient for depolymerization of the carbohydrate polymers, i.e., hemicellulose, to occur. Solids are then separated from liquids to produce a liquid hydrolysate and residual solids.

Nonlimiting examples of pressure changing devices that may be used for feeding acid impregnated biomass into a digestor in the methods described herein include plug screw feeders, rotary valves, or lockhopper arrangements. In one embodiment, the acid impregnated biomass material is heated in the digestor with direct steam. In another embodiment, the acid impregnated biomass material is heated in the digestor with indirect steam.

In some embodiments, the digestor is operated under pressure. In one embodiment, the digestor is a continuous feed, pressure rated, screw conveyor vessel.

In some embodiments, acid impregnated biomass material is fed to the digestor at a liquid to solid ratio of about 1:1 to about 20:1.

At commercial scale, about 100 to about 15,000 ODMT (oven dry metric tonnes) of acid impregnated feedstock may be fed to the digestor per day.

The conditions in the digestor may be adjusted to produce a residual solid product with desired structural characteristics (i.e., length of resulting cellulosic fiber).

In one embodiment, the biomass is treated with nitric acid at a concentration of about 0.1% (w/w) to about 0.5% (w/w), or about 0.5% (w/w) to about 1.0% (w/w), the digestor is operated at a pressure of about 90 to about 110 psig, a temperature of about 160° C. to about 167° C., or about 167° C. to about 176° C., or about 176° C. to about 185° C. and a residence time of about 3 to about 20, about 8 to about 20, or about 5 to about 10 minutes, and the residual solid material contains cellulosic fiber that is at least about 0.35 or 0.37 mm in length.

In one embodiment, the biomass is treated with nitric acid at a concentration of about 0.05% to about 4% (w/w), or about 1% (w/w) to about 1.5% (w/w), digestor is operated at a pressure of about 90 to about 110 psig, a temperature of about 160° C. to about 185° C. or about 60° C. to about 180° C., and a residence time of about 4 to about 15, or about 5 to about 10 minutes, and the residual solid material contains cellulosic fiber that is less than about 0.35, 0.30, or 0.28 mm in length.

After the digestion process, solids are separated from liquids. Nonlimiting examples of methods for such separation include a screw press, belt filter press, centrifuge, settling tank, vacuum filter, or rotary drum dryer.

The liquid hydrolysate typically contains about 10 to about 150 g/l hemicellulose sugar molecules depending on the amount of moisture in the feed and the water added. For example, the hydrolysate may contain mannose, xylose, glucose, arabinose, and/or galactose. In some embodiments, some depolymerization of the cellulose portion may also occur, for example, up to about 10%.

### Conditioning of Hydrolyzed Feedstock

In some embodiments, hydrolysate, produced as described herein is “conditioned” to remove inhibitors of microbial growth and/or bioproduct, production. Such inhibitors may include, but are not limited to, organic acids, furans, phenols, soluble lignocellulosic materials, extractives, and ketones. Inhibitors present in wood hydrolysates may include, but are not limited to, 5-hydroxyy-methyl furfural (HMF), furfural, aliphatic acids, levulinic acid, acetic acid, formic acid, phenolic compounds, vanillin, dihydroconiferylalcohol, coniferyl aldehyde, vanillic acid, hydroquinone, catechol, acetoguaiacone, homovanillic acid, 4-hydroxy-benzoic acid, Hibbert's ketones, ammonium nitrate and/or other salts, p-coumaric acid, ferulic acid, 4-hydroxybenzoic acid, vanillic acid, syringaldehyde, and glucuronic acid.

Nonlimiting examples of conditioning processes include vacuum or thermal evaporation, overliming, precipitation, adsorption, enzymatic conditioning (e.g., peroxidase, laccase), chemical conversion, distillation, evaporation, filtration, and ion exchange, or a combination thereof. In one embodiment, conditioning includes contact of hydrolyzed feedstock with an ion exchange resin, such as an anion or cation exchange resin. Inhibitors may be retained on the resin. In one embodiment, the ion exchange resin is an anion exchange resin. Ion exchange resins may be regenerated with caustic, some solvents, potentially including those generated in the bioproduct, e.g., biofuel, production processes described herein, or other known industrial materials. In other embodiments, inhibitors may be precipitated by a metal salt (for example, a trivalent metal salt, for example, an aluminum or iron salt, such as aluminum sulfate or ferric chloride), and/or a flocculant such as polyethylene oxide or other low density, high molecular weight polymers.

In one embodiment, hydrolysate is conditioned on ion exchange resin, such as an anion exchange resin, e.g., Duolite A7, at acidic pH, for example, pH about 2.5 to about 5.5, about 3.5 to about 4.5, or about 2.5, 3, 3.5, 4, 4.5, 5, or 5.5.

In one embodiment, hydrolysate is conditioned with a metal salt, for example, a trivalent metal salt, such as an aluminum or iron salt, e.g., aluminum sulfate or ferric chloride. In some embodiments, the metal salt is added at a concentration of about 1 g/L to about 6 g/L, or about 3 g/L to about 5 g/L. In some embodiments, the pH is adjusted with a base to a basic pH, such as about 9.5 to about 11, or about 9.5, 10, 10.5, or 11, for example, with ammonium hydroxide or ammonia gas.

In some embodiments, microbial growth and/or bioproduct, e.g., biofuel, titer, yield, and/or productivity is increased when conditioned hydrolyzed feedstock is used, in comparison to identical hydrolyzed feedstock which has not been subjected to the conditioning process.

In some embodiments, a microorganism that is tolerant to inhibitors in hydrolyzed feedstock is used, or the microorganism used for bioproduct production develops increased tolerance to inhibitors over time, e.g., by repeated passaging, rendering the conditioning step unnecessary or uneconomical.

### Compositions

Hydrolysates and conditioned hydrolysates, prepared in accordance with any of the methods described herein, are provided. Such hydrolysate compositions may be used in a downstream process such as microbial fermentation to produce one or more bioproduct(s) of interest. The hydrolysate provides soluble sugar molecules which may be used as a carbon source to support microbial growth.

Biomass material from which hemicellulose has been extracted, prepared in accordance with any of the methods described herein, is also provided. In some embodiments, the biomass material contains visible cellulosic fiber, for example, fiber that is at least about 0.35 or 0.37 mm in length. Such a material may hydrolyzed, for example, via acid hydrolysis, to release soluble cellulosic sugars, and the cellulosic hydrolysate thus produced may be used, for example, as a carbon source to support microbial growth for production of bioproduct(s) of interest. Alternatively, this material may be subjected to a pulping process to make pulp from the cellulose, and then used as a raw material, for example, for production of paper, rayon, absorbent material, etc. In some embodiments, the biomass material contains cellulosic fiber that is less than about 0.35, 0.30, or 0.28 mm in length, for example, without or with very little visible cellulosic fiber. Such a material may be hydrolyzed, for example, via enzymatic or acid hydrolysis, and used as a carbon source in a microbial fermentation for production of bioproduct(s). Alternatively, this material may be used as a fuel, for example, pelleted and burned.

### Methods for Producing a Bioproduct

Methods are provided for producing a bioproduct. The methods include culturing a microorganism in a medium that contains hydrolysate or conditioned hydrolysate, prepared as described herein, as a soluble sugar source to support microbial growth for production of one or more bioproduct(s) of interest. In some embodiments, microbial growth and/or bioproduct titer, yield, and/or productivity may be increased when conditioned hydrolyzed feedstock, prepared as described herein, is used in a microbial fermentation process, in comparison to identical hydrolyzed feedstock which has not been subjected to the conditioning process.

In some embodiments, the bioproduct is a biofuel, for example, butanol, acetone, and/or ethanol. In some embodiments, the bioproduct is solvent (e.g., a polar protic or aprotic solvent), biomolecule, organic acids, alcohols, fatty acid, aldehyde, lipid, long chain organic molecule, vitamin, or sugar alcohol.

### Fermentation

The methods for bioproduct production herein include fermentation of a bioproduct-producing microorganism in a bioreactor in a growth medium that contains hydrolysate or conditioned hydrolysate prepared according to any of the methods described herein.

In some embodiments, the bioproduct production includes fermentation of a bioproduct-producing microorganism in an immobilized cell bioreactor (i.e., a bioreactor containing cells that are immobilized on a support, e.g., a solid support). In some embodiments, an immobilized cell bioreactor provides higher productivity due to the accumulation of increased productive cell mass within the bioreactor compared with a stirred tank (suspended cell) bioreactor. In some embodiments, the microbial cells form a biofilm on the support and/or between support particles in the growth medium.

In some embodiments, the bioproduct production process herein includes continuous fermentation of a microorganism (continuous addition of conditioned hydrolyzed feedstock and withdrawal of product stream). Continuous fermentation minimizes the unproductive portions of the fermentation cycle, such as lag, growth, and turnaround time, thereby reducing capital cost, and reduces the number of inoculation events, thus minimizing operational costs and risk associated with human and process error.

Fermentation may be aerobic or anaerobic, depending on the requirements of the bioproduct-producing microorganism.

In some embodiments, an immobilized bioproduct-producing Clostridium strain is fermented anaerobically in a continuous process as described herein.

One or more bioreactors may be used in the bioproduct production systems and processes described herein. When multiple bioreactors are used they can be arranged in series and/or in parallel. The advantages of multiple bioreactors over one large bioreactor include lower fabrication and installation costs, ease of scale-up production, and greater production flexibility. For example individual bioreactors may be taken off-line for maintenance, cleaning, sterilization, and the like without appreciably impacting the production schedule. In embodiments in which multiple bioreactors are used, the bioreactors may be run under the same or different conditions.

In a parallel bioreactor arrangement, hydrolyzed feedstock is fed into multiple bioreactors, and effluent from the bioreactors is removed. The effluent may be combined from multiple bioreactors for recovery of the bioproduct, or the effluent from each bioreactor may be collected separately and used for recovery of the bioproduct.

In a series bioreactor arrangement, hydrolyzed feedstock is fed into the first bioreactor in the series, the effluent from the first bioreactor is fed into a second downstream bioreactor, and the effluent from each bioreactor in the series is fed into the next subsequent bioreactor in the series. The effluent from the final bioreactor in the series is collected and may be used for recovery of the bioproduct.

Each bioreactor in a multiple bioreactor arrangement can have the same species, strain, or mix of species or strains of microorganisms or a different species, strain, or mix of species or strains of microorganisms compared to other bioreactors in the series.

Immobilized cell bioreactors allow higher concentrations of productive cell mass to accumulate and therefore, the bioreactors can be run at high dilution rates, resulting in a significant improvement in volumetric productivity relative to cultures of suspended cells. Since a high density, steady state culture can be maintained through continuous culturing, with the attendant removal of product containing fermentation broth, smaller capacity bioreactors can be used. Bioreactors for the continuous fermentation of C. acetobutylicum are known in the art. (U.S. Pat. Nos. 4,424,275, and 4,568,643.)

Numerous methods of fermentor inoculation are possible including addition of a liquid seed culture to the bottom or the top of the bioreactor and recirculation of the media to encourage growth throughout the bed. Other methods include the addition of a liquid seed culture or impregnated solid support through a port located along the reactor's wall or integrated and loaded with the solid support material. Bioreactor effluent may also be used to inoculate an additional bioreactor and in this case any residual fermentable materials may be converted in the secondary reactor, increasing yield/recovery.

In a similar manner, support material may be added to the reactor through bottom, top, or side loading to replenish support material that becomes degraded or lost from the bioreactor.

### Fermentation Media

Fermentation media for the production of bioproduct contain feedstock, e.g., a hydrolyzed or conditioned hydrolyzed feedstock, prepared as described herein, as a source of fermentable carbohydrate molecules.

As known in the art, in addition to an appropriate carbon source, fermentation media must contain suitable nitrogen source(s), mineral salts, cofactors, buffers, and other components suitable for the growth of the cultures and promotion of the enzymatic pathway necessary for the production of the desired bioproduct. In some embodiments, salts and/or vitamin B12 or precursors thereof are included in the fermentation media. In some cases, hydrolyzed feedstock may contain some or all of the nutrients required for growth, minimizing or obviating the need for additional supplemental material.

The nitrogen source may be any suitable nitrogen source, including but not limited to, ammonium salts, yeast extract, corn steep liquor (CSL), and other protein sources including, but not limited to, denatured proteins recovered from distillation of fermentation broth or extracts derived from the residual separated microbial cell mass recovered after fermentation. Phosphorus may be present in the medium in the form of phosphate salts, such as sodium, potassium, or ammonium phosphates. Sulfur may be present in the medium in the form of sulfate salts, such as sodium or ammonium sulfates. Additional salts include, but are not limited to, magnesium sulfate, manganese sulfate, iron sulfate, magnesium chloride, calcium chloride, manganese chloride, ferric chloride, ferrous chloride, zinc chloride, cupric chloride, cobalt chloride, and sodium molybdate. The growth medium may also contain vitamins such as thiamine hydrochloride, biotin, and para-aminobenzoic acid (PABA). The growth medium may also contain one or more buffering agent(s) (e.g., MES), one or more reducing agent(s) (e.g., cysteine HCl), and/or sodium lactate, which may serve as a carbon source and pH buffer.

### Microorganisms

The systems and processes described herein include one or more microorganism(s) that is (are) capable of producing one or more bioproduct(s) of interest. In embodiments in which two or more microorganisms are used, the microorganisms may be the same or different microbial species and/or different strains of the same species.

In some embodiments, the microorganisms are bacteria or fungi. In some embodiments, the microorganisms are a single species. In some embodiments, the microorganisms are a mixed culture of strains from the same species. In some embodiments, the microorganisms are a mixed culture of different species. In some embodiments, the microorganisms are an environmental isolate or strain derived therefrom.

In some embodiments of the processes and systems described herein, different species or strains, or different combinations of two or more species or strains, are used in different bioreactors with different conditioned hydrolyzed feedstocks as a carbohydrate source.

In some embodiments, a fungal microorganism is used, such as a yeast. Examples of yeasts include, but are not limited to, Saccharomyces cerevisiae, S. bayanus, S. carlsbergensis, S. Monacensis, S. Pastorianus, S. uvarum and Kluyveromyces species. Other examples of anaerobic or aerotolerant fungi include, but are not limited to, the genera Neocallimastix, Caecomyces, Piromyces and other rumen derived anaerobic fungi.

In some embodiments, a bacterial microorganism is used, including Gram-negative and Gram-positive bacteria. Non-limiting examples of Gram-positive bacteria include bacteria found in the genera of Staphylococcus, Streptococcus, Bacillus, Mycobacterium, Enterococcus, Lactobacillus, Leuconostoc, Pediococcus, and Propionibacterium. Non-limiting examples of specific species include Enterococcus faecium and Enterococcus gallinarium. Non-limiting examples of Gram-negative bacteria include bacteria found in the genera Pseudomonas, Zymomonas, Spirochaeta, Methylosinus, Pantoea, Acetobacter, Gluconobacter, Escherichia and Erwinia.

In one embodiment, the bacteria are Clostridium species, including but not limited to, Clostridium saccharobutylicum, Clostridium acetobutylicum, Clostridium beijerinckii, Clostridium puniceum, and environmental isolates of Clostridium.

Further examples of species of Clostridium contemplated for use in this invention can be selected from C. aurantibutyricum, C. butyricum, C. cellulolyticum, C. phytofermentans, C. saccharolyticum, C. saccharoperbutylacetonicum, C. tetanomorphum, C. thermobutyricum, C. thermocellum, C. puniceum, C. thermosaccharolyticum, and C. pasterianum.

Other bacteria contemplated for use in the processes and systems herein include Corynebacteria, such as C. diphtheriae, Pneumococci, such as Diplococcus pneumoniae, Streptococci, such as S. pyogenes and S. salivarus, Staphylococci, such as S. aureus and S. albus, Myoviridae, Siphoviridae, Aerobic Spore-forming Bacilli, Bacilli, such as B. anthracis, B. subtilis, B. megaterium, B. cereus, Butyrivibrio fibrisolvens, Anaerobic Spore-forming Bacilli, Mycobacteria, such as M. tuberculosis hominis, M. bovis, M. avium, M. paratuberculosis, Actinomycetes (fungus-like bacteria), such as, A. israelii, A. bovis, A. naeslundii, Nocardia asteroides, Nocardia brasiliensis, the Spirochetes, Treponema pallidium, Treponema pertenue, Treponema carateum, Borrelia recurrentis, Leptospira icterohemorrhagiae, Leptospira canicola, Spirillum minus, Streptobacillus moniliformis, Trypanosomas, Mycoplasmas, Mycoplasma pneumoniae, Listeria monocytogenes, Erysipelothrix rhusiopathiae, Streptobacillus monilformis, Donvania granulomatis, Bartonella bacilliformis, Rickettsiae, Rickettsia prowazekii, Rickettsia mooseri, Rickettsia rickettsiae, and Rickettsia conori. Other suitable bacteria may include Escherichia coli, Zymomonas mobilis, Erwinia chrysanthemi, and Klebsiella planticola.

In some embodiments, the microorganisms are from the genera Clostridium, Enterococcus, Klebsiella, Lactobacillus, Enterococcus, Escherichia, Pichia, Pseudomonas, Synechocystis, Saccharomyces, or Bacillus. In some embodiments, the microbial strain is a Clostridium species, for example, Clostridium acetobutylicum, Clostridium beijerinckii, Clostridium saccharobutylicum, Clostridium puniceum, Clostridium saccharoperbutylacetonicum, Clostridium pasteuranium, Clostridium butylicum, Clostridium aurantibutyricum, Clostridium tetanomorphum, Clostridium thermocellum, and Clostridium thermosaccharolyticum. In some embodiments, the microorganisms are obligate anaerobes. Non-limiting examples of obligate anaerobes include Butyrivibrio fibrosolvens and Clostridium species.

In other embodiments, the microorganisms are microaerotolerant and are capable of surviving in the presence of small concentrations of oxygen. In some embodiments, microaerobic conditions include, but are not limited, to fermentation conditions produced by sparging a liquid media with a gas of at least about 0.01% to at least 5% or more O₂ (e.g., 0.01%, 0.05%, 0.10%, 0.50%, 0.60%, 0.70%, 0.80%, 1.00%, 1.20%, 1.50%, 1.75%, 2.0%, 3%, 4%, 5% or more O₂). In another aspect, the microaerobic conditions include, but are not limited to, culture conditions with at least about 0.05 ppm dissolved O₂ or more (e.g., 0.05, 0.075, 0.1, 0.15, 0.2, 0.3, 0.4, 0.5, 0.6, 0.8, 1.0, 2.0, 3.0, 4.0, 5.0, 8.0, 10.0, ppm or more).

Alternatively, parent strains can be isolated from environmental samples such as wastewater sludge from wastewater treatment facilities including municipal facilities and those at chemical or petrochemical plants. The latter are especially attractive as the isolated microorganisms can be expected to have evolved over the course of numerous generations in the presence of high product concentrations and thereby have already attained a level of desired product tolerance that may be further improved upon.

Parent strains may also be isolated from locations of natural degradation of naturally occurring feedstocks and compounds (e.g., a woodpile, a saw yard, under fallen trees, landfills). Such isolates may be advantageous since the isolated microorganisms may have evolved over time in the presence of the feedstock and thereby have already attained some level of conversion and tolerance to these materials that may be further improved upon.

Individual species or mixed populations of species can be isolated from environmental samples. Isolates, including microbial consortiums can be collected from numerous environmental niches including soil, rivers, lakes, sediments, estuaries, marshes, industrial facilities, etc. In some embodiments, the microbial consortiums are strict anaerobes. In other embodiments, the microbial consortiums are obligate anaerobes. In some embodiments, the microbial consortiums are facultative anaerobes. In still other embodiments, the microbial consortiums do not contain species of Enterococcus or Lactobacillus.

When mixed populations of specific species or genera are used, a selective growth inhibitor for undesired species or genera can be used to prevent or suppress the growth of these undesired microorganisms.

### Culture Conditions

Optimal culture conditions for various industrially important microorganisms are known in the art. As required, the culture conditions may be anaerobic, microaerotolerant, or aerobic. Aerobic conditions are those that contain oxygen dissolved in the media such that an aerobic culture would not be able to discern a difference in oxygen transfer with the additional dissolved oxygen, and microaerotolerant conditions are those where some dissolved oxygen is present at a level below that found in air or air saturated solutions and frequently below the detection limit of standard dissolved oxygen probes, e.g., less than 1 ppm. The cultures can be agitated or left undisturbed. Typically, the pH of the media changes over time as the microorganisms grow in number, consume feedstock and excrete organic acids. The pH of the media can be modulated by the addition of buffering compounds to the initial fermentation media in the bioreactor or by the active addition of acid or base to the growing culture to keep the pH in a desired range. Growth of the culture may be monitored by measuring the optical density, typically at a wavelength of 600 nm, or by other methods known in the art.

Clostridium fermentations are generally conducted under anaerobic conditions. For example, ABE fermentations by C. acetobutylicum are typically conducted under anaerobic conditions at a temperature in the range of about 25° C. to about 40° C. Historically, suspension cultures did not use agitators, but relied on evolved or sparged gas to mix the contents of the bioreactors. Cultures, however, can be agitated to ensure more uniform mixing of the contents of the bioreactor. For immobilized cultures, a bioreactor may be run without agitation in a fixed bed (plug flow) or fluidized/expanded bed (well-mixed) mode. Thermophilic bacterial fermentations can reach temperatures in the range of about 50° C. to about 80° C. In some embodiments, the temperature range is about 55° to about 70° C. In some embodiments, the temperature range is about 60° C. to about 65° C. For example, Clostridium species such as C. thermocellum or C. thermohydrosulfuricum may be grown at about 60° C. to about 65° C. The pH of the Clostridium growth medium can be modulated by the addition of buffering compounds to the initial fermentation media in the bioreactor or by the active addition of acid or base to the growing culture to keep the pH in a desired range. For example, a pH in the range of about 3.5 to about 7.5, or about 5 to about 7, may be maintained in the medium for growth of Clostridium.

### Immobilization of Microorganisms on Solid or Semi-Solid Support

In some embodiments, microorganisms are grown immobilized on a solid or semi-solid support for production of one or more bioproduct(s) of interest.

Immobilization of the microorganism, from spores or vegetative cells, can be by any known method. In one embodiment, entrapment or inclusion in the support is achieved by polymerizing or solidifying a spore or vegetative cell containing solution. Useful polymerizable or solidifiable solutions include, but are not limited to, alginate, ic-carrageenan, chitosan, polyacrylamide, polyacrylamide-hydrazide, agarose, polypropylene, polyethylene glycol, dimethyl acrylate, polystyrene divinyl benzene, polyvinyl benzene, polyvinyl alcohol, epoxy carrier, cellulose, cellulose acetate, photocrosslinkable resin, prepolymers, urethane, and gelatin.

In another embodiment, the microorganisms are incubated in growth medium with a support. Useful supports include, but are not limited to, bone char, cork, clay, resin, sand, porous alumina beads, porous brick, porous silica, celite (diatomaceous earth), polypropylene, polyester fiber, ceramic, (e.g., porous ceramic, such as porous silica/alumina composite), lava rock, vermiculite, ion exchange resin, coke, natural porous stone, macroporous sintered glass, steel, zeolite, engineered thermal plastic, concrete, glass beads, Teflon, polyetheretherketone, polyethylene, wood chips, sawdust, cellulose fiber (pulp), or other natural, engineered, or manufactured products. The microorganisms may adhere to the support and form an aggregate, e.g., a biofilm.

In another embodiment, the microorganism is covalently coupled to a support using chemical agents like glutaraldehyde, o-dianisidine (U.S. Pat. No. 3,983,000), polymeric isocyanates (U.S. Pat. No. 4,071,409), silanes (U.S. Pat. Nos. 3,519,538 and 3,652,761), hydroxyethyl acrylate, transition metal-activated supports, cyanuric chloride, sodium periodate, toluene, or the like. See also U.S. Pat. Nos. 3,930,951 and 3,933,589.

In one embodiment, immobilized spores, such as those of Clostridium, e.g., C. acetobutylicum, are activated by thermal shock and then incubated under appropriate conditions in a growth medium whereby vegetative growth ensues. These cells remain enclosed in or on the solid support. After the microorganisms reach a suitable density and physiological state, culture conditions can be changed for bioproduct production. If the immobilized cells lose or exhibit reduced bioproduct production ability, they can be reactivated by first allowing the cells to sporulate before repeating the thermal shock and culture sequence.

Vegetative cells can be immobilized in different phases of their growth. For microorganisms that display a biphasic culture, such as C. acetobutylicum with its acidogenic and solventogenic phases, cells can be immobilized after they enter the desired culture phase in order to maximize production of the desired products, where in the case of C. acetobutylicum it is the organic acids acetic acid and butyric acid in the acidogenic phase and the solvents acetone, butanol and ethanol in the solventogenic phase. Alternatively, biphasic cells can be immobilized in the acidogenic phase and then adapted for solvent production.

In some embodiments, microorganisms to be immobilized in a bioreactor are introduced by way of a cell suspension. Generally, these microorganisms are dispersed in the media as single cells or small aggregates of cells. In other embodiments, the microorganisms are introduced into the bioreactor through the use of suspended particles that are colonized by the microorganisms. These suspended particles can be absorbed onto the solid support and frequently are of sufficiently small size that they can enter and become immobilized in the pore structures of the solid support. Typically, regardless of the suspended particle size, microorganisms can be transferred by contact with the solid support. A biofilm on the introduced particles can transfer to and colonize these new surfaces. In some embodiments, the desired characteristics of the microorganisms can only be maintained by culturing on a solid support, thereby necessitating the use of small colonized particle suspensions for seeding a solid support in a bioreactor.

### Support for Immobilized Microbial Growth

In some embodiments, a bioproduct producing microorganism is grown in an immobilized form on a solid or semi-solid support material in a bioreactor as described herein. In some embodiments, the support contains a porous material. Non-limiting examples of suitable support materials include bone char, synthetic polymers, natural polymers, inorganic materials, and organic materials.

Natural polymers include organic materials such as cellulose, lignocellulose, hemicellulose, and starch. Organic materials include feedstock such as plant residue and paper. Composites of two or more materials may also be used such as mixtures of synthetic polymer with natural plant polymer.

Examples of semi-solid media include alginate, K-carrageenan and chitosan, polyacrylamide, polyacrylamide-hydrazide, agarose, polypropylene, polyethylene glycol, dimethyl acrylate, polystyrene divinyl benzene, polyvinyl benzene, polyvinyl alcohol, epoxy carrier, cellulose, cellulose acetate, photocrosslinkable resin, prepolymers, urethane, and gelatin. Examples of solid support include cork, clay, resin, sand, porous alumina beads, porous brick, porous silica, celite, wood chips or activated charcoal.

Suitable inorganic solid support materials include inorganic materials with available surface hydroxy or oxide groups. Such materials can be classified in terms of chemical composition as siliceous or nonsiliceous metal oxides. Siliceous supports include, inter alia, glass, colloidal silica, wollastonite, cordierite, dried silica gel, bentonite, and the like. Representative nonsiliceous metal oxides include alumina, hydroxy apatite, and nickel oxide.

In some embodiments, the support material is selected from bone char, polypropylene, steel, diataomaceous earth, zeolite, ceramic, (e.g., porous ceramic, such as porous silica/alumina composite), engineered thermal plastic, clay brick, concrete, lava rock, wood chips, polyester fiber, glass beads, Teflon, polyetheretherketone, polyethylene, vermiculite, ion exchange resin, cork, resin, sand, porous alumina beads, coke, natural porous stone, macroporous sintered glass, or a combination thereof. In one embodiment, the support material is bone char. Useful support material has a high surface area to volume ratio such that a large amount of active, productive cells can accumulate in the bioreactor. Useful supports may contain one or more macrostructured components containing one or more useful support material(s) that promotes good fluidmechanical properties, for example, a wire mesh/gauze packing material used for traditional distillation tower packing.

In some embodiments, the support material includes a surface area of at least about 100 m²/m³. In some embodiments, the support material comprises a bulk density of at least about 0.15 g/cm³. In some embodiments, the support material comprises a ball-pan hardness number of at least about 60. In some embodiments, the support material comprises a yield strength of at least about 20 MPa.

The particle size for the support material will vary depending upon bioreactor configuration and operation parameters. In some embodiments, the support material is sized by sieving. In some embodiments, the particles are classified by the sieve number of the mesh that they can pass through. In some embodiments, the particles are sieved with a mesh that has a U.S. Sieve Number of 3½, 4, 5, 6, 7, 8, 10, 12, 14, 16, 18, 20, 25, 30, 35, 40, 45, 50, 60, or 70. In some embodiments, the particles are sieved at least twice, first using a mesh with larger openings followed by a mesh with smaller openings to yield particles within a defined particle size distribution range. In some embodiments, the particles are at least about 100 μm, 200 μm, 300 μm, 400 μm, 500 μm, 600 μm, 700 μm, 800 μm, 900 μm, 1000 μm, 1,100 μm, 1,200 μm, 1,300 μm, 1,400 μm, 1,500 μm, 1,600 μm, 1,700 μm, 1,800 μm, 1,900 μm, 2,000 μm, 3,000 μm, 4,000 μm, 5,000 μm, 6,000 μm, 7,000 μm, 8000 μm, 9,000 μm, 10,000 μm, 12,500 μm, 15,000 μm, 17,500 μm, 20,000 μm, 22,500 μm, or 25,000 μm in diameter. In some embodiments, the particles are less than about 100 μm, 200 μm, 300 μm, 400 μm, 500 μm, 600 μm, 700 μm, 800 μm, 900 μm, 1000 μm, 1,100 μm, 1,200 μm, 1,300 μm, 1,400 μm, 1,500 μm, 1,600 μm, 1,700 μm, 1,800 μm, 1,900 μm, 2,000 μm in diameter. In further embodiments, at least about 80%, 85%, 90%, 95%, or 100% of the particle have diameters that are in the range of about 100-400 μm, 100-600 μm, 100-800 μm, 200-500 μm, 200-800 μm, 200-1000 μm, 400-800 μm, 400-1000 μm, 500-1000 μm, 600-1,200 μm, 800-1,400, μm 1,000-1,500, μm 1,000-2000 μm, 2,000-4,000 μm, 4,000-6,000 μm, 5,000-12,000 μm, 3,000-15,000 μm, or 6,000-25,000 μm. In some embodiments, the particle diameters are the equivalent diameters, a parameter that takes into account the irregular shapes of the individual particles.

Ideally, the semi-solid or solid support material should have a high surface area. This can be achieved through the use of small sized particles, particles with high porosity, or a combination thereof. In some embodiments, the surface area of the particles is at least about 0.003 m²/g, 0.01 m²/g, 0.02 m²/g, 0.05 m²/g, 0.1 m²/g, 0.5 m²/g, 1 m²/g, 5 m²/g, 10 m²/g, 25 m²/g, 50 m²/g, 75 m²/g, 100 m²/g, 125 m²/g, 150 m²/g, 175 m²/g, 200 m²/g, 225 m²/g, 250 m²/g, 275 m²/g, 300 m²/g, 325 m²/g, 350 m²/g, 375 m²/g, 400 m²/g, 425 m²/g, 450 m²/g, 500 m²/g, 600 m²/g, 700 m²/g, 800 m²/g, 900 m²/g, 1000 m²/g, or 2000 m²/g. Additionally, the bulk density should be sufficiently high so that the smallest particles settle out of the fluid stream in the column expansion zone and/or particle disengagement zone and are thereby retained in the bioreactor. In some embodiments, the bulk density of the support is at least about 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, 1.2 g/cm³, or 1.3 g/cm³. The support material should have sufficient hardness to resist abrasion and thereby avoid appreciable dust formation when the support particles touch or collide with each other. In some embodiments, the support has a ball-pan hardness number of at least about 20, 40, 60, 80, 100, 120, 140, 160 or 200. The support material should also have sufficient tensile strength to resist shattering due to internal stresses, which may be caused by the growth of biofilms inside support material pores. In some embodiments, the support has a yield strength of at least about 20 MPa, 40 MPa, 60 MPa, 80 MPa, 100 MPa, 120 MPa, 140 MPa, 160 MPa, 180 MPa, 200 MPa, 300 MPa, or 400 MPa. The support material should also have the ability to resist being crushed by the accumulated weight of material above it. Crush strength is another measurement of the mechanical strength of the support and is typically a function of the composition, shape, size, and porosity of the material (increase in port volume may negatively impact particle strength). In some embodiments, the crush strength is at least about 8 kg.

In some embodiments, the support material is chosen to support growth of the fermenting bioproduct producing microorganism as a biofilm. The biofilm may grow on exterior surfaces of support particles, in the fluid space between support particles, and/or on surfaces in the interior of pores of the support material.

### Continuous Process

In some embodiments, a continuous process for bioproduct production is provided. In a continuous production process herein, a carbohydrate-containing feedstock hydrolysate or conditioned hydrolysate containing soluble sugar molecules, prepared according to any of the methods described herein, is continuously fed to one or more bioreactors for microbial production of the bioproduct, the bioproduct is continuously produced by immobilized microorganism(s) in the one or more bioreactors, and bioproduct-containing effluent, i.e., fermentation broth, is continuously withdrawn from the one or more reactors, for the duration of fermentation. In some embodiments, feedstock is continuously hydrolyzed to release soluble sugar molecules, and continuously conditioned prior to introduction of the conditioned hydrolyzed feedstock into the bioreactor(s). The conditioning process may operate continuously downstream from a feedstock hydrolysis process, and upstream from the bioreactor(s), and conditioned hydrolyzed feedstock may be continuously fed to the bioreactor for the duration of fermentation. In some embodiments, the microorganism is tolerant to inhibitors and the conditioning step is not used. In one embodiment, the feedstock is lignocellulosic feedstock, and is hydrolyzed with nitric acid to release soluble sugar molecules from cellulose and hemicellulose, as described supra.

In some embodiments, the continuous process may also include downstream continuous concentration and/or purification processes for recovery of the bioproduct, wherein continuously withdrawn effluent is continuously processed in one or more concentration and/or purification processes to produce a bioproduct.

In some embodiments, the process may also include deconstruction of the feedstock and/or removal of extractives from the feedstock, as described herein. Deconstruction and/or removal of extractives may be continuous or may occur prior to or periodically throughout the continuous process.

In some embodiments, the process operates continuously for at least about 50, 100, 200, 300, 400, 600, 800, 1000, 1350, 1600, 2000, 2500, 3000, 4000, 5000, 6000, 7000, 8000, or 8400 hours.

A “continuous” process as described herein may include periodic or intermittent partial or complete shutdowns of one or more parts of the bioproduct production system for processes such as maintenance, repair, regeneration of resin, etc.

Continuous fermentation, with constant feed of feedstock and withdrawal of product-containing microbial broth, can minimize the unproductive portions of a fermentation cycle, such as lag, growth, and turnaround time, thereby reducing the capital cost, and can reduce the number of inoculation events, thus minimizing operational costs and risk associated with human and process error.

The continuous methods and systems described herein can utilize one or more, e.g., one, two, or three or more, bioreactors. When multiple (two or more) bioreactors are used, they may be arranged in parallel, series, or a combination thereof. The bioreactors can grow the same or different strains of microorganism(s).

The following examples are intended to illustrate, but not limit, the invention.

### EXAMPLES

### Example 1

Grey stage Lodgepole pine chips, moisture content approximately 24.9%, were screened for debris and passed through a thermomechanical disintegrator in order to ensure (1) adequate acid impregnation throughout the chip for the liberation of hemicellulosic sugars, and (2) to remove some wood extractives.

The disintegrator was a Bauer/Andritz RT Impressifiner, used under the following conditions. Some dilution water was added to saturate the wood chips, steam was added at a delivery pressure of 1.38 bar, residence time was 20 seconds, and the flow restriction at the exit of the RT Impressifiner was set to 1 inch.

A sample of the preliminary pressate was collected. 1.42% (w/w) nitric acid was added to the solid material at the exit of the RT Impressifiner and resulted in a 32-37% (w/w) solids stream. The material was collected in drums, stored at about 10° C. for processing 12-18 hours later. The temperature of the material at the exit of the disintegrator was 60° C., and cooled about 15-20° C. in 15 hours.

The acid impregnated material was then added to a feed hopper for a digestor feeding system. The digestor was a continuous feed, pressure rated, screw conveyor vessel operated nominally at 7.92-6.13 bar (90-110 psig), which corresponds to a steam saturation temperature of 167-176° C. Material was fed at an average rate of 11 ODMT/day to the ˜1000 L digestor through a plug screw feeder (PSF) system with a compression ratio of approximately 8:1 or a rotary valve. The liquids to solids ratio feeding the digestor was 2.1:1. The residence time within the digestor was 300-480 seconds.

The liquid pressate from the PSF was measured at a rate of approximately 2 gallons per minute (gpm) (7.6 liters/minute) and contained free nitric acid (pH 1.3), as well as turpentine/tall oil type components (by smell). In some cases, all of the liquid pressate was added back to the digestor. In other cases, a portion of the liquid pressate was added back to the digestor with the balance of the 2 gallons per minute supplied by city water. In other cases, the PSF pressate was discarded and 2 gallons per minute of water were added to the digestor.

Pressure was maintained in the digestor with a 6 inch ball type blow valve. The hydrolysate and residual solids were expanded to atmospheric pressure through a cyclone to separate the vapor from the liquid and solids. Some volatiles were removed in the vent stream. Residual solids were approximately 32% by weight.

A 560 screw press was used to attempt to separate solids from liquids. Very little dewatering was achieved. Average feed solids was measured at about 36% and the residual solids exiting from the screw press was measured to be 36-37%, due to the small average fiber dimension.

Surprisingly, the residual material had very little fiber quality or structure. Microscopic imaging of the residual material showed little distinguishable cellulosic fiber. The fiber had the following characteristics:

| | |
|---|---|
| Length weighted average length (mm) | 0.276 |
| Arithmetic average length (mm) | 0.151 |
| Weight weighted average length (mm) | 0.544 |
| Average width (μm) | 36.14 |
| Surface area (m²/kg) | 1441 |
| Fiber Classifications | |
| % on 14 mesh | 0.5 |
| % on 28 mesh | 1.7 |
| % on 48 mesh | 3.7 |
| % on 100 mesh | 8.2 |
| % on 200 mesh | 9.6 |
| % through 200 mesh | 76.3 |

*% denotes weight fraction retained on the indicated mesh.

The hydrolysate liquor contained significant concentrations of primarily hemicellulose sugars (˜75 g/L) in the ratios typical of softwood dilute acid hydrolysis: mannose, xylose, glucose, arabinose, and galactose.

In a follow up experiment, material that had been passed through the disintegrator under conditions of either no acid added or 1.42% (w/w) nitric acid was reacted in a 7.6 liter Parr bomb type reactor. No additional water was added, in order to duplicate as closely as possible the conditions in the digestor (5 minutes, 166° C.). In this run, 750 g of the moist feed (36.8% solids by weight) were added. 450 g of water were also added to the reactor. Live steam was added until the reactor reached the setpoint temperature at which point the blow valve was released (5 minutes) and the material was blown into a blow tank where the pressure was permitted to equilibrate with the environment.

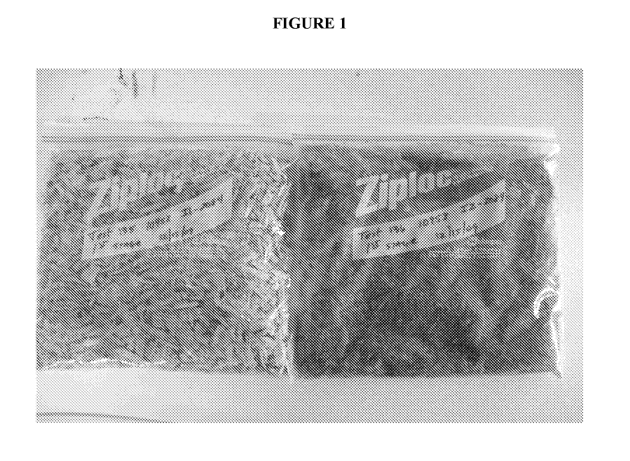

The results are shown in FIG. 1. The residual solids from the no acid condition are shown in the photograph on the left, and the residual solids produced with 1.42% nitric acid are shown in the photograph on the right. Visible cellulosic fiber is exhibited in the no acid sample but not in the nitric acid sample.

### Example 2

Grey stage Lodgepole pine chips, moisture content approximately 31.6%, were passed through a thermomechanical disintegrator, as described in Example 1 except that the wood chips were not screened for debris and the flow restriction at the exit of the disintegrator was 0.5 inch.

0.44% (w/w) nitric acid was added to the solid material at the exit of the RT Impressifiner and resulted in a 33.0% (w/w) solid discharge. The material was fed to a digestor as described in Example 1, with storage from 1 to 12 hours prior to processing. The digestor conditions were as described in Example 1, except the residence time was 360 seconds. No PSF pressate was retained in the process, but water was added to the rotary feeder (˜1.9 gpm) parallel to the PSF, the mechanical refiner (post digestor, between the digestor and the blow valve) (˜3 gpm), and the discharge cyclone, which is located post blow valve.

The resulting visible fiber quality was greater than in the product described in Example 1, and was effectively dewatered in the screw press. Residual solids were 57.7% by weight.

The fiber had the following characteristics:

| | |
|---|---|
| Length weighted average length (mm) | 0.379 |
| Arithmetic average length (mm) | 0.202 |
| Weight weighted average length (mm) | 0.862 |
| Average width (μm) | 49.72 |
| Surface area (m²/kg) | 1105 |
| Fiber Classifications | |
| % on 14 mesh | 11.6 |
| % on 28 mesh | 18.0 |
| % on 48 mesh | 19.7 |
| % on 100 mesh | 20.2 |
| % on 200 mesh | 7.4 |
| % through 200 mesh | 23.1 |

*% denotes weight fraction retained on the indicated mesh.

The hydrolysate liquor contained significant concentrations of hemicellulose sugars (˜43.5 g/L) in the ratios typical of softwood dilute acid hydrolysis: mannose, xylose, glucose, arabinose, and galactose.

### Example 3

Clostridium was grown anaerobically in a packed bed bioreactor with 1 L nominal volume and 670 mL working volume. The L/D ratio of the bioreactor was 3.

The Clostridium was immobilized on bonechar. The bonechar particles had a size of 3000 to 5000 microns, with a bulk density of about 0.72/ml. About 1.5 pounds of bonechar was loaded into the reactor. Immobilization was achieved by first filling the reactor with about 670 mL of CP3 media with 6% w/v softwood sugars synthetic mix (20.04% w/w D-glucose, 31.32% w/w D-xylose, 12.88% w/w L-arabinose, 35.76% w/w D-mannose) and then adding to the reactor 60 mL of Clostridium broth that had an OD at 600 nm of about 0.8, and recirculating the contents of the reactor for 24 hours.

The initial growth medium as well as the medium used during the continuous part of the fermentation, contained conditioned beetle killed lodgepole pine acid hydrolysate with about 45 g/L sugar, supplemented with P2 medium components and trace elements, except that ammonium was added as ammonium sulfate instead of as ammonium acetate. The hydrolysate was prepared as described in Example 1, and conditioned on Duolite A7 resin at acidic pH.

Continuous culture was started around 21 hours after inoculation by pumping the growth media at a constant rate into the bottom of the bioreactor and continuously removing broth from the top of the bioreactor in order to maintain a constant liquid level in the bioreactor. Continuous fermentation continued for 422 hours.

The feed rate for the run was 8 g/min and N₂ was added at a rate of 0.1 L/min for the duration of the fermentation. During the fermentation period between 164 and 422 hours the average pH was about 5.1. The average butanol titer, productivity, and yield were 7.6 g butanol/L, 5.5 g butanol/L/hr, and 0.26 g butanol/g carbohydrate, respectively.

### Example 4

Clostridium was grown anaerobically in a packed bed bioreactor with 111.3 L nominal volume and 65.7 L working volume. The L/D ratio of the bioreactor was 5.7.

The Clostridium was immobilized on bonechar initially screened with a 5×8 mesh, with a bulk density of about 45 lb/ft3. About 100 pounds of bonechar was loaded into the reactor. Immobilization was achieved by first filling the reactor with about 100 L of fermentation media with 4% by weight softwood hydrolysate, prepared as described in Example 2 and conditioned on Duolite A7 resin at acidic pH, draining approximately 15 L of feed media and then adding to the reactor about 15 L L of Clostridium broth that had A600 absorbance of about 1. The fermentation broth was circulated for approximately 24 h prior to setting the reactor into continuous operation.

Continuous culture was achieved after the bioreactor had been inoculated by pumping the growth media at a constant rate into the bottom of the bioreactor and continuously removing broth from the top of the bioreactor in order to maintain a constant liquid level in the bioreactor.

The feed rate for the run was about 540 g/min. The average pH was about 5.5 and the average pressure was about 3.4 psi. N₂ was added at a rate of 1.0 L/min for the duration of the fermentation. After 106 hours elapsed fermentation time, yield of butanol, acetone, ethanol, acetic acid, and butyric acid were 0.220, 0.050, 0.020, 0.015, and 0.111 g/g sugars converted, respectively. Sugar conversion in the reactor varied throughout the run and was approximately 50-80%.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention which is delineated by the appended claims.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A method for deconstructing and extracting sugar molecules from biomass that comprises cellulose and hemicellulose, comprising:
(a) mechanically disintegrating said biomass in the presence of water and under a first pressure, thereby producing liquid and/or vapor and solid disintegrated biomass;
(b) separating the liquid and/or vapor from the solid disintegrated biomass, wherein step (b) may be performed after or in conjunction with step (a);
(c) contacting the solid disintegrated biomass with acid in an amount sufficient to depolymerize a polymeric carbohydrate component of the biomass, thereby producing acid impregnated disintegrated biomass;
(d) feeding the acid impregnated disintegrated biomass into a digestor through a pressure changing device, wherein said acid impregnated disintegrated biomass is heated under a second pressure in said digestor at a temperature and for an amount of time sufficient to permit the depolymerization reaction to occur, wherein a composition is produced that comprises a liquid hydrolysate and residual solids; and
(e) separating the liquid hydrolysate and residual solids, wherein the liquid hydrolysate comprises hemicellulose sugar molecules and the residual solids comprise cellulosic fiber.

2. A method according to claim 1, wherein the method is performed with an acid concentration in step (c), a residence time in step (d), and a temperature in step (d) sufficient to produce a liquid hydrolysate that comprises hemicellulose sugar molecules and residual solids that comprise cellulosic fiber that is at least about 0.35 mm in length.

3. A method according to claim 1, wherein the method is performed with an acid concentration in step (c), a residence time in step (d), and a temperature in step (d) sufficient to produce a liquid hydrolysate that comprises hemicellulose sugar molecules and residual solids that comprise cellulosic fiber that is less than about 0.35 mm in length.

4. A method according to claim 3, wherein the acid concentration in step (c) is about 1% (w/w) to about 1.5% (w/w), the residence time in step (d) is about 5 minutes to about 10 minutes and the temperature in step (d) is about 60° C. to about 180° C.

5. A method according to claim 1, wherein the liquid and/or vapor that is separated from solid disintegrated biomass in step (b) comprises extractives.

6. A method according to claim 1, wherein said pressure changing device is selected from a plug screw feeder, a rotary valve, or a lockhopper arrangement.

7. A method according to claim 1, wherein said heating of the acid impregnated disintegrated biomass in the digestor is with direct or indirect steam.

8. A method according to claim 1, wherein said mechanical disintegrating is performed in a thermo-mechanical disintegrator.

9. A method according to claim 8, wherein said thermo-mechanical disintegrator is selected from a modular screw device, an oil press, and a screw press.

10. A method according to claim 1, wherein mechanical disintegration is performed at a first pressure and a residence time sufficient to shear apart the biomass to make it accessible for acid-catalyzed depolymerization of carbohydrate polymers.

11. A method according to claim 10, wherein said first pressure is about 5 to about 50 psig and said residence time is about 5 to about 60 seconds.

12. A method according to claim 1, wherein the acid is nitric acid.

13. A method according to claim 2, wherein the acid is nitric acid at a concentration of about 0.1% (w/w) to about 0.5% (w/w).

14. A method according to claim 3, wherein the acid is nitric acid at a concentration of about 0.05% (w/w) to about 4% (w/w).

15. A method according to claim 1, wherein the digestor is operated under a second pressure of about 50 to about 150 psig.

16. A method according to claim 1, wherein the second pressure is higher than the first pressure.

17. A method according to claim 1, wherein the digestor is a continuous feed, pressure rated, screw conveyor vessel.

18. A method according to claim 1, wherein the material that is fed to the digestor comprises a liquid to solid ratio of about 1:1 to about 20:1.

19. A method according to claim 2, wherein the digestor is operated at a second pressure of about 90 to about 110 psig, a temperature of about 167° C. to about 176° C. and a residence time of about 3 to about 20 minutes.

20. A method according to claim 3, wherein the digestor is operated at a second pressure of about 90 to about 110 psig, a temperature of about 120° C. to about 180° C. and a residence time of about 4 to about 15 min.

21. A method according to claim 1, wherein solids are separated from liquids in step (e) in a screw press, belt filter press, centrifuge, settling tank, vacuum filter, a sieve screen, or rotary drum dryer.

22. A method according to claim 1, wherein the liquid hydrolysate comprises about 10 to about 150 g/l sugar molecules.

23. A method according to claim 22, wherein the sugar molecules comprise mannose, xylose, glucose, arabinose, and galactose.

24. A method according to claim 1, wherein the biomass is lignocellulosic material selected from softwood and hardwood, or a combination thereof.

25. A method according to claim 24, wherein the lignocellulosic material is in the form of wood chips.

26. A deconstructed biomass material from which hemicellulose sugars have been extracted and which retains cellulosic fiber that is at least about 0.35 mm in length, wherein said material is prepared according to claim 2.

27. A deconstructed biomass material from which hemicellulose sugars have been extracted and which does not retain visible cellulosic fiber that is at least about 0.35 mm in length, wherein said material is prepared according to claim 3.

28. A method according to claim 1, further comprising conditioning said liquid hydrolysate to remove one or more inhibitor of microbial growth and/or production of a bioproduct, thereby producing a conditioned hydrolysate.

29. A conditioned hydrolysate produced according to claim 28.

30. A method for producing a bioproduct, comprising culturing a microorganism that produces said bioproduct in a medium that comprises a conditioned hydrolysate according to claim 29 under conditions suitable for production of said bioproduct.

31. A method according claim 30, wherein said bioproduct is a biofuel selected from butanol, ethanol, and acetone, or a combination thereof.

32. A method for deconstructing biomass that comprises cellulose and hemicellulose for the extraction of sugar molecules from said biomass, comprising:
(a) contacting the biomass with acid in an amount sufficient to depolymerize a polymeric carbohydrate component of the biomass, thereby producing acid impregnated disintegrated biomass;
(b) feeding the acid impregnated disintegrated biomass into a digestor through a pressure changing device, wherein said acid impregnated disintegrated biomass is heated under pressure in said digestor at a temperature and for an amount of time sufficient to permit the depolymerization reaction to occur, wherein a composition is produced that comprises a liquid hydrolysate and residual solids; and
(c) separating the liquid hydrolysate and residual solids,
wherein the liquid hydrolysate comprises hemicellulose sugar molecules and the residual solids comprise cellulosic fiber that is less than about 0.35 mm in length.

33. A method according to claim 32, wherein the acid concentration in step (a) is about 0.1% (w/w) to about 5% (w/w), the residence time in step (b) is about 8 to about 20 minutes, and the temperature in step (b) is about 160° C. to about 180° C.

34. A method according to claim 32, wherein the acid is nitric acid.

35. A method according to claim 32, wherein the digestor is operated under a pressure of about 50 to about 150 psig.

36. A biomass material from which hemicellulose sugars have been extracted and which does not retain cellulosic fiber that is at least about 0.35 mm in length, wherein said material is prepared according to claim 32.

37. A method according to claim 32, further comprising conditioning said liquid hydrolysate to remove one or more inhibitor of microbial growth and/or production of a bioproduct, thereby producing a conditioned hydrolysate.

38. A conditioned hydrolysate produced according to claim 37.

39. A method for producing a bioproduct, comprising culturing a microorganism that produces said bioproduct in a medium that comprises a conditioned hydrolysate according to claim 38 under conditions suitable for production of said bioproduct.

40. A method according claim 39, wherein said bioproduct is a biofuel selected from butanol, ethanol, and acetone, or a combination thereof.
